(19) United States
(12) Patent Application Publication
(10) Pub. No.: US 2018/0147021 A2
(43) Pub. Date: May 31, 2018

(54) HOLDER FOR A MARKER DEVICE

(71) Applicant: Brainlab AG, Feldkirchen (DE)

(72) Inventors: Oliver Fleig, Baldham (DE); Melanie Stulpe, Rosenheim (DE)

(21) Appl. No.: 15/105,429

(22) PCT Filed: Dec. 20, 2013
(86) PCT No.: PCT/EP2013/077631

§ 371 Date: Jun. 16, 2016

(51) Int. Cl. A61B 90/00

(52) U.S. Cl. CPC A61B 90/39

(57) ABSTRACT

The present invention relates to a medical fastening system for fastening a medical marker device to an anatomical body part, the fastening system comprising:
a) a base part comprising a holding unit for holding the marker device; and
b) a connecting part (5) for connecting the base part to the anatomical body part, characterized in that
c) the base part comprises a fastening unit for fastening the connecting part to the base part so that the base part remains rotatable relative to the connecting part.

### RELATED APPLICATION DATA

This application is a national phase application of International Application No. PCT/EP2013/077631 filed Dec. 20, 2013 published in the English language.

The present invention is directed to a medical fastening system for fastening a medical marker device to an anatomical body part and a medical system comprising the medical fastening system and the medical marker device. The fastening system is in particular a holder for a marker device such as a reference star.

In navigated medical procedures such as navigated surgery, it is a common necessity to attach a marker device to an anatomical body part such as a bone. Known solutions for this problem include holders comprising a base part which comprises a tightening screw for fastening the holder to pins which have been fastened to the anatomical body part beforehand. This solution, however, means that a specific step of attaching the holder to the pins. Furthermore, operating the tightening screw causes comparably large forces to be exerted on the pins and the anatomical body part, respectively. Thus, the risk of injury and/or undesirable deformation or dislocation of the pins arises.

FR 2867376 A1 US 2005/020920 AI, US 2004/068263 AI, EP 1 803 413 A2, US 2010/063511 A1, US 2004/167391 A1, US 2011/118750 A1, and U.S. Pat. No. 6,203,543 B1 disclose devices for fastening a medical marker device to an anatomical body part which allow to rotate at least a part of the device relative to a connecting part used to fasten the device to the anatomical body part. inherent to those devices is, however, that careless operation may lead to an undesired change of the relative position between the marker device and the anatomical body part.

A problem to the solved by the present invention therefore is to provide a fastening system for fastening a medical marker device to an anatomical body part which can be efficiently operated and avoids undesired movement of the fastening system relative to the anatomical body part.

This problem is solved by the subject-matter of any appended independent claim. Advantages, advantageous features, advantageous embodiments and advantageous aspects of the present invention are disclosed in the following and contained in the subject-matter of the dependent claims. Different advantageous features can be combined in accordance with the invention wherever technically expedient and feasible. Specifically, a feature of one embodiment which has the same or a similar function to another feature of another embodiment can be exchanged with said other feature. A feature of one embodiment which adds an additional function to another embodiment can in particular be added to said other embodiment.

### Exemplary Short Description of the Present Invention

In the following, a short description of the specific features of the present invention is given which shall not be understood to limit the invention only to the features or combination of features described in this section.

The present invention provides a fastening system for fastening a marker device such as a reference star to an anatomical body part. The fastening system comprises for example a block-shaped anchor serving as a base part and two for example pin-shaped parts serving as a fastening unit and a fixing unit, respectively, of the fastening system, for fastening and fixing, respectively, the anchor to the anatomical body part. The marker device can be attached to the anchor before or after fixing the anchor to the anatomical body part. According to a notable feature of the invention, the anchor remains rotatable around the fastening unit (the fastening pin) after the pin has been fastened to the anatomical body part for example by inserting it into a previously drilled hole in a bone representing the anatomical body part.

### General Description of the Invention

In this section, a description of the general, in particular preferred features of the present invention is given.

In order to solve the aforementioned problem, in particular a medical fastening system for fastening a medical marker device to an anatomical body part is provided which comprises the following preferred features. Preferably, the medical fastening system comprises a base part which comprises a holding unit for holding the marker device. The base part is in particular an anchor which serves to carry and stabilize the position of the marker device relative to the anatomical body part. The anatomical body part comprises at least one of hard tissue (for example a bony structure or cartilage) and soft tissue (such as skin tissue or muscle tissue). The base part preferably has an at least substantially cylindrical shape or an at least substantially cuboid shape (for example the shape of a cuboid with rounded corners).

Preferably the base part comprises a polymer material. Alternatively or additionally, the base part may comprise a metal material such surgical steel or titanium. In view of a desired weight reduction of the base part, it is however preferred that it is at least substantially almost completely made of a polymer.

The holding unit according to a preferred embodiment has the shape of a recess such as a cylindrical cutout into the interior of a base part which is open towards the upper surface of the base part. According to a further preferred embodiment, the holding unit has the shape of a through-hole through an upper sidewall of the base part, wherein the sidewall has a finite thickness. According to an even further preferred embodiment, the holding unit is embodied by an integral connection between the base part and the marker device. In particular, the marker device and the base part may be shaped as one integral structure, i.e. integrally.

Furthermore, the fastening system preferably comprises a connecting part for connecting the base part of the anatomical body part. According to a preferred embodiment, the connecting part comprises, in particular is embodied by, a pin. Preferably, the pin is removably attached to the connecting part. According to a different, also preferred embodiment, the connecting part cannot be removed from the base part. However, it is preferred in all embodiments that the connecting part and the base part are movable relative to one another. In particular, it is desirable that the connecting part is able to rotate relative to the base part. Preferably, the connecting part is inserted into and/or through the base part. For example, the connecting part is pin-shaped and disposed in a through-hole through the base part. Alternatively, the connecting part is pin-shaped and inserted in to a blind hole of the base part which opens towards the lower surface of the base part.

Preferably, the connecting part comprises at least one translation limiting means which limits translatability of the base part relative to the connecting part. For example, the translation limiting means also serves to secure the connecting part against removal from the base part, for example by a radial extension such as a disc-shaped surface which extends around and in a radial direction of the pin (i.e. forms a radially enlarged section of the pin) and is configured to abut the upper surface of the base part or a radially enlarged portion of the blind hole. In particular in case the pin is inserted through a through-hole of the base part, the pin preferably comprises an abutment piece which also extends in a radial direction around the pin and is configured to abut the lower surface of the base part. Thus, the pin can be held in a predetermined position relative to the base part at least with finite position. The pin preferably has an outer thread at its lower end, i.e. the end of the pin which points away from the lower surface of the base part and the marker device if the marker is attached to the base part.

Preferably, the base part comprises a fastening unit for fastening the connecting part to the base part so that the base part is rotatable relative to the connecting part. Two embodiments of the fastening unit are the aforementioned through-hole and the blind hole. Alternatively the fastening unit may comprise an interior thread which is configured to interact with an exterior thread on a for example pin-shaped connecting part. According to a further embodiment, the fastening unit comprises a clamping means, and the connecting part comprises a longitudinal positional means. The longitudinal positioning means of the connecting part comprises preferably a plurality of engagement notches which neighbour each other in the longitudinal direction of a pin-shaped connecting part. Alternatively, the longitudinal positioning means comprises a roughened exterior surface in the longitudinal direction of the connecting part. If the longitudinal positioning means is embodied by the engagement notches, the clamping means is embodied by preferably a corresponding counter-surface of a biased part of the fastening unit which is configured to press against the reference surface of the pin as to hold the fastening unit in a fixed position in the longitudinal direction of the pin. In particular if the longitudinal positioning means is embodied by the plurality of notches, the clamping means is preferably embodied by a pawl which is biased into its closing position so that it can engage into the notches so as to fix the position of the fastening means relative to the pin in the longitudinal direction of the pin. The function of the clamping means therefore is to interact with the longitudinal positioning means for fixing the positioning of the fastening unit relative to the connecting part in the longitudinal direction of the connecting part. In addition thereto, the fastening unit advantageously comprises a releasing means for releasing the fastening unit from the connecting part, in particular for releasing the clamping means from the longitudinal positioning means. For example, the releasing means comprises a button which is attached to the clamping means and can be operated by a user to press the clamping means in its opening direction so as to release the engagement between the clamping means and the longitudinal positioning means. After doing so, the fastening unit can be moved relative to the connecting part, in particular in the longitudinal direction of the connecting part. In particular in this configuration, the fastening unit is configured to keep the base part translatable relative to the connecting part (in the longitudinal direction of the connecting part) after the connecting part has been fastened to the base part. If, however, the biasing force (which can be exerted by for example a spring) onto the biased part is rather low, the releasing means (in particular the button) is not required. Rather, the clamping means may be configured to open upon e.g. manual force applied to e.g. the base part for removing it from the longitudinal positioning means.

Preferably, the connecting part is at least substantially a longitudinally-shaped structure, in particular at least one of a screw, a nail and a pin. Further preferably, the connecting part comprises a metal material such as surgical steel or titanium so as to ensure mechanical stability. Further preferably, the connecting part is configured to be attached to the marker device in particular. The connecting part may comprise an upper end (for example in the case where the medical connecting part is inserted into a through-hole of the base part) to which the marker device can be fastened. For example, the connecting part may comprise an upper portion which fits into a fastening mechanism (e.g. a clipping mechanism) of the marker device. The connecting part then preferably also comprises a resisting unit for resisting a force applied to the marker device after the marker device has been attached to the connecting part. Such a resisting unit can be embodied by for example a locking mechanism such as a clipping mechanism which allows to clip to the marker device onto the upper end of the connecting part. This avoids removal of the marker device from the connecting part and therefore the medical fastening system by accidently touching it for example during a surgical procedure.

Preferably, the base part comprises a fixing unit for fixing the base part to the anatomical part. The fixing unit preferably is not identical to the fastening unit. In particular the fixing unit and the fastening unit constitute two different parts of the base part and fulfil different functions. Fixing in this context means that the position of the base part relative to the anatomical body part is fixed in three dimensions and in particular no rotation of the base part relative to the anatomical body part and/or preferably also relative to the connecting part is possible anymore if the fixing unit is used as envisaged (in particular is activated). According to a preferred embodiment, the fixing unit comprises, in particular is embodied by, a through-hole through the base part which is preferably located in a position which is diametrical relative to the position of the fastening unit. The through-hole may also comprise an interior thread and therefore be a thread hole. Alternatively, the fixing unit comprises a clamping mechanism for clamping a fixation means such as another pin, another nail or another screw. In particular, the fixation means is not identical to the connecting part. According to a further preferred embodiment, the fixation means comprises an exterior thread which is configured to interact with the interior thread of the fixing unit. The clamping mechanism for clamping the fixation means may take the shape of for example at least one clamping arm, preferably two clamping arms for clamping the fixation means.

The invention furthermore relates to a medical system comprising the above-described fastening system and the medical marker device. The marker device and the fastening system (in particular the marker device and the holding unit) may be formed integrally (i.e. as one coherent, in particular unitary, part) or separately, i.e. as two individual parts which during use of the inventive fastening system can be attached to one another. Thus, the medical system may be delivered as one piece or as individual parts, in particular also by different suppliers and/or manufacturers.

The medical fastening system of the invention is sought to be used as follows:
1. Two holes are drilled into the anatomical body part, the holes being spaced by a distance which is equal to the spacing between the fastening unit and the fixing unit.
2. The base part with the connecting part attached to it is fastened to a first one of the two holes, in particular by inserting the lower end of the connecting part into that hole (in particular screwed into the hole if the connecting part has an external thread).
3. Since the base part is configured to remain rotatable relative to the connecting part, the base part can then be rotated around the connecting part (irrespective of how deep the connecting part has been screwed into the first hole) so that the fixing unit (in particular a second through-hole through the base part or at least two clamping arms) are positioned above the second hole. The fixation means can then be inserted into the fixing unit and for example screwed into the second hole in the anatomical body part so as to fix the position of the base part relative to the anatomical body part.

Depending on whether the fastening system and the marker device are provided as an integral structure or separate structures, the marker device can finally be attached to the holding unit or it can be pre-fastened to the holding unit so that it is automatically attached to the anatomical body part by fixing the base part to the anatomical body part.

### Definitions

In this section, definitions for specific terminology used in this disclosure are offered which also form part of the present disclosure.

It is the function of a marker to be detected by a marker detection device (for example, a camera or an ultrasound receiver or analytical devices such as CT or MRI) in such a way that its spatial position (i.e. its spatial location and/or alignment) can be ascertained. The detection device is in particular part of a navigation system. The markers can be active markers. An active marker can for example emit electromagnetic radiation and/or waves which can be in the infrared, visible and/or ultraviolet spectral range. The marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

A marker can in particular take the shape of a two-dimensional graphical pattern which is attached to a preferably planar surface. Such a pattern comprises, in particular consists of, for example a Moire pattern which is detectable in a digital image acquired by a digital imaging device such as a digital video camera or a digital still camera.

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is in particular known to a navigation system and is for example stored in a computer of the navigation system.

A “reference star” refers to a device with a number of markers, advantageously three markers, attached to it, wherein the markers are (in particular detachably) attached to the reference star such that they are stationary, thus providing a known (and advantageously fixed) position of the markers relative to each other. The position of the markers relative to each other can be individually different for each reference star used within the framework of a surgical navigation method, in order to enable a surgical navigation system to identify the corresponding reference star on the basis of the position of its markers relative to each other. It is therefore also then possible for the objects (for example, instruments and/or parts of a body) to which the reference star is attached to be identified and/or differentiated accordingly. In a surgical navigation method, the reference star serves to attach a plurality of markers to an object (for example, a bone or a medical instrument) in order to be able to detect the position of the object (i.e. its spatial location and/or alignment). Such a reference star in particular features a way of being attached to the object (for example, a clamp and/or a thread) and/or a holding element which ensures a distance between the markers and the object (in particular in order to assist the visibility of the markers to a marker detection device) and/or marker holders which are mechanically connected to the holding element and which the markers can be attached to.

### DESCRIPTION OF THE FIGURES

In the following, an example embodiment will be described by reference to the figures, wherein the invention shall not be limited to the features described in connection with the figures, and wherein

FIGS. 1a to 1c show two aspects of a first embodiment of the invention;

FIGS. 2a to 2b show further views of the first embodiment;

FIGS. 3a to 3b show a second embodiment of the invention;

FIGS. 4a to 4e show a base part according to a third embodiment of the invention which comprises a clamping means and a releasing means.

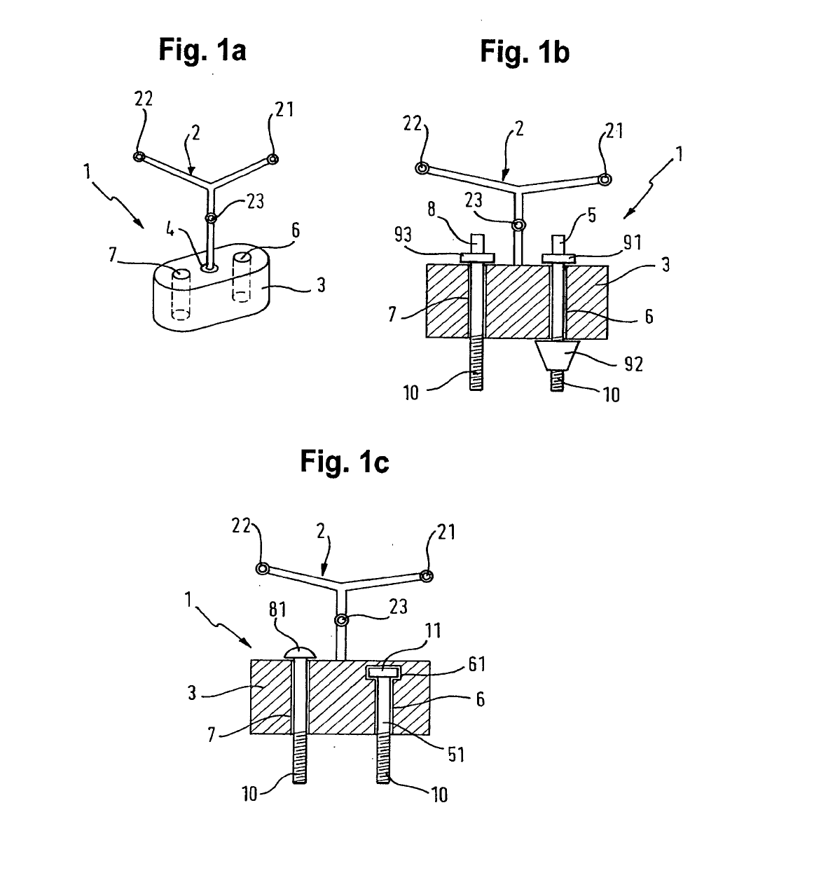

FIG. 1a shows a medical fastening system 1 comprising a base part 3 to which a marker device 2 is attached via a holding unit 4. For example, the holding part of the marker device 2 comprises an outer thread which is screwed into an inner thread of the holding unit 4 which is embodied by a blind hole in the upper surface of the base part 3. In an alternative embodiment, the holding part of the marker device 2 comprises a magnet and the holding unit 4 is embodied by a recess also comprising a magnet in its interior, for example at its bottom surface. The holding part is inserted into the recess and held in position in the holding unit 4 by the attraction force between the magnets. The marker device 2 is embodied by a reference star having three markers 21, 22, 23 in a predetermined spatial relationship relative to one another. The base part 3 comprises a fastening unit 6 embodied by a through-hole and a fixing unit 7 embodied by another through-hole through the base part.

FIGS. 1b and 1c illustrate a cross-section through the base part 3 shown in FIG. 1a along a plane in which the longitudinal extensions of the fastening unit 6 and the fixing unit 7 lie.

As shown in FIG. 1b, a connecting part 5 embodied by a pin having a thread 10 at its lower end is inserted into the fastening unit 6. The connecting part 5 has translation limiting means 91, 92 at its positions which are configured to lie just above or below, respectively, the upper and lower surface, respectively of the base part 3 when the pin is inserted into the through-hole 6. The translation limiting means 91, 92 are embodied by radial extensions which extend radially from the connecting parts 5 (the pin). The base part 3 is able to rotate relative to the connecting part 5 when the connecting part 5 is inserted into the fastening unit 6. For finally fixing the fastening system 1 relative to the anatomical body part, a fixation means 8 embodied by another pin is inserted into the blind hole 6 so that the disc-shaped translation limiting means 11 lies in the radial clearance 61 and is able to abut the lower limiting surface of the radial clearance 61 so that the pin 51 cannot be pulled out of the blind hole 6. In this aspect of the first embodiment, the fixation means is embodied by a screw 81 which is inserted into the fixing unit 7 embodied by a through-hole. It is notable that the aspects of the FIGS. 1b and 1c can be combined in the sense that a fastening unit 6 embodied by the blind hole of FIG. 1c can also be combined with a pin-like fixation means 8 as shown in FIG. 1b. Likewise, a fastening unit 6 embodied by a through-hole as shown in FIG. 1c can also be combined with a screw-shaped fixation means 81 as shown in FIG. 1c.

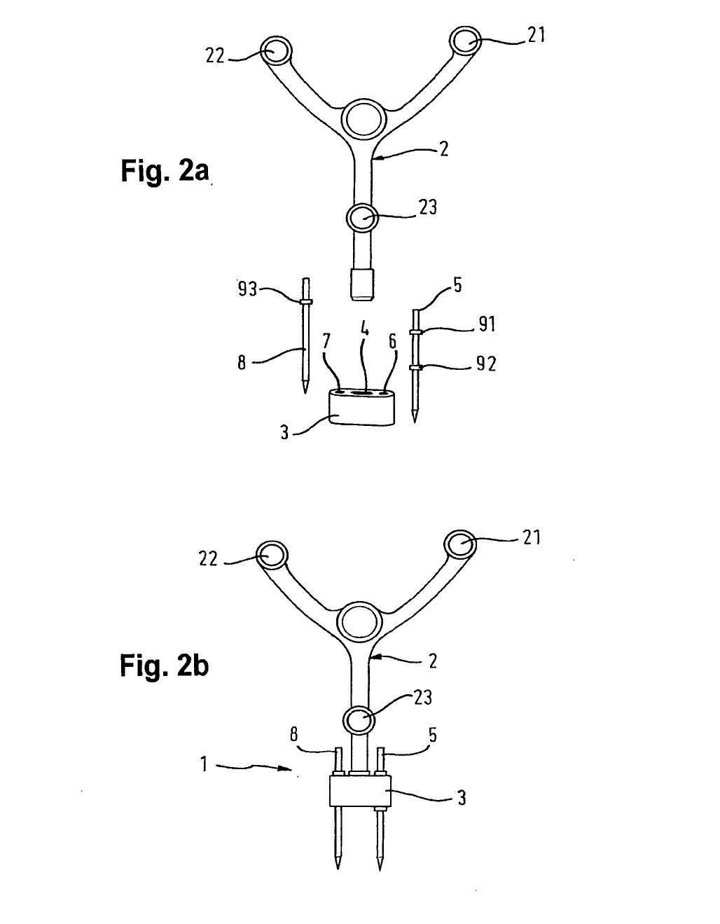

FIG. 2a shows another aspect of the fastening system 1 in accordance with a first embodiment. A marker device embodied by reference star 2 is inserted into holding unit 4 of the base part 3 which comprises two through-holes as a fastening unit 6 and a fixing unit 7, respectively. A pin-shaped connecting part having translation limiting means 91, 92 can be inserted into the fastening unit 6, and a pin-shaped fixation means 8 having a translation limiting means 93 can be inserted in to the fixing unit 7. Within the framework of the invention, also an embodiment is envisaged in which the fixation means 8 does not have the translation limiting means 93.

FIG. 2b gives an illustration of the assembled system comprising the marker device 2 and the fastening system 1.

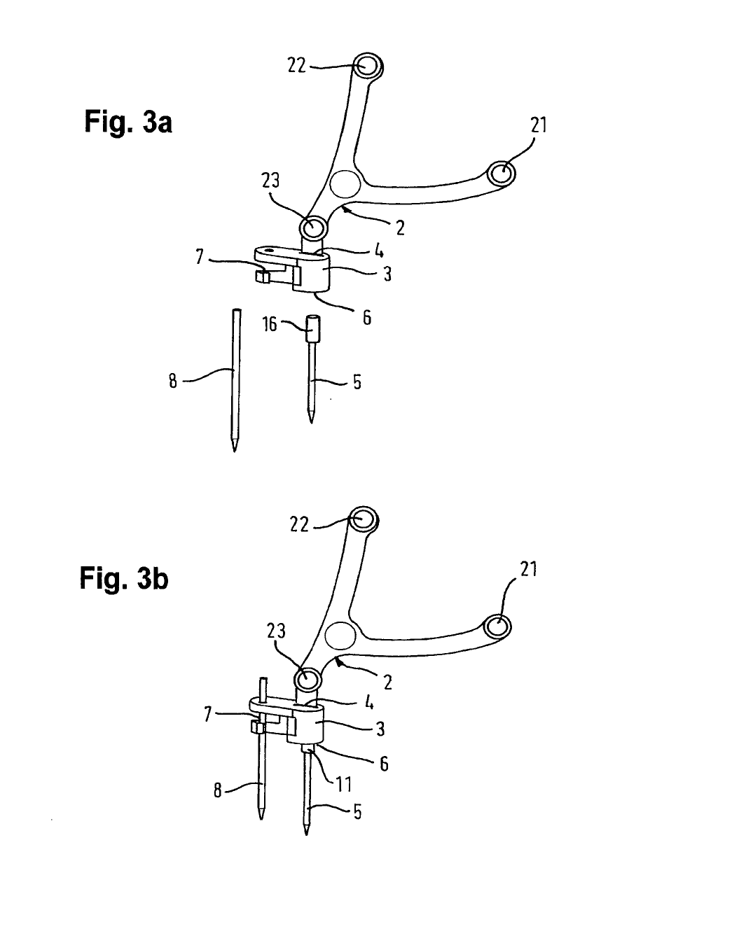

In the embodiment shown in FIGS. 3a and 3b, the marker device 2 and the holding part 4 are formed integrally and also form one integral part with the base part 3. The base part 3 (and therefore in a sense also the marker device 2) can be clipped onto the connecting part 5 having a clip inset 16 at its upper end for insertion into the base part 3. In this sense, the fastening unit 6 serves also for fastening the marker device 2 to the connecting part 5. In particular, the clip inset 16 can be inserted into the fastening unit 6. The base part 3 also comprises two (in particular biased) holding arms as a fixing unit 7 which are configured to clamp the pin-shaped fixation means 8. When the base part 3 is attached to the connecting part 5 by positioning the fastening unit 6 on the inset 16, it remains rotatable relative to the connecting part 5. Thus, when the marker device 2 has been positioned on the connecting part 5 which is in turn attached to the anatomical body part, the marker device 2 can be rotated until the holding arms 7 are positioned over the second drilled hole for inserting the fixation means 8. After positioning then in this manner, the pin-shaped fixation means 8 can be pushed through the biased holding arm 7 and into the second drilled hole.

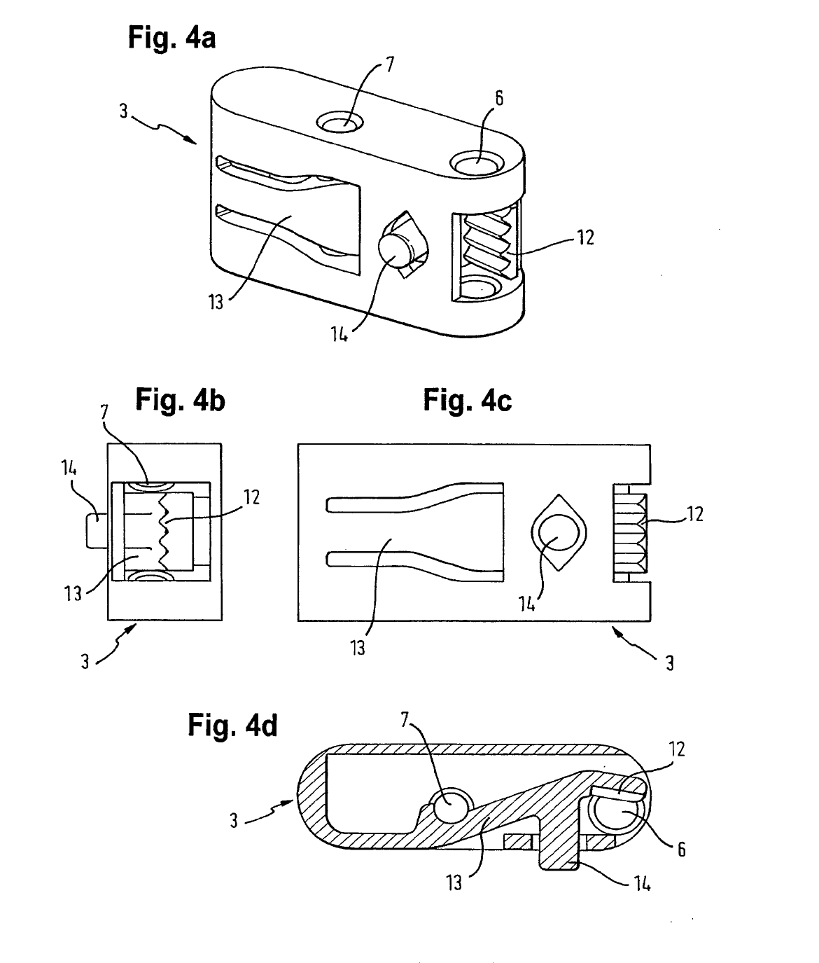

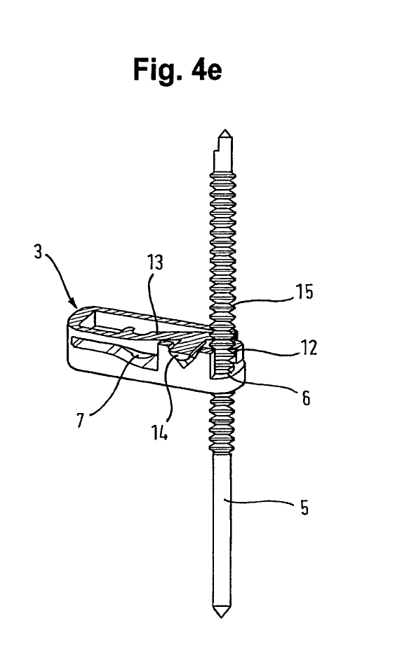

FIGS. 4a to 4e show base part 3 according to a further embodiment which has a clamping means embodied by a biased arm 13 disposed in the interior of the base part 3 and having teeth 12 at its distal end. The biased arm 13 is biased when it clamps the connecting part 5. The biased arm 13 is an embodiment of the biasing means for biasing a clamping means which is configured to fix the base part 3 to a connecting part 5. The biased arm 13 is disposed in the interior of the base part 3 such that it leaves a clearance for the axes defined by the through-holes in the upper end lower surface, respectively, the base part 7 which define the fastening unit 6 and the fixing unit 7, respectively. The distal end of the biased arm 13 having the teeth 12 on it is biased towards the axes defined by the through-hole of the fastening unit 6 such that the teeth 12 can engage with a positioning means 15 embodied by notches on a for example pin-shaped connecting part 5 as shown in FIG. 4e. The base part shown in FIG. 4d is a horizontal section through a base part 3 as shown in FIGS. 4a to 4e along a plane which cuts the releasing means 14 in half. The releasing means 14 is embodied by a button which can be pressed by a user so as to move the teeth 12 out of engagement with the positioning means 15 so that the base part 3 can be moved up and down along the longitudinal axis of the connecting part 5 while the connecting part 5 is inserted into the fastening unit 6. As becomes clear from FIG. 4d, the releasing means 14 can be activated (i.e. the button can be pressed) only if no fixation means 8 is inserted into the fixing means 7. Otherwise, the fixation means 8 would hamper movement of the biased arm 13 towards the interior of the base part 3. This supports safety aspects. In particular, the base part 3 cannot be released from the connecting part 5 while a fixation means 8 is inserted into the second drilled hole on the anatomical body part so that the position of the base part 3 relative to the fixation means 8 and the connecting part 5 is fixed while the fixation means 8 is inserted into the fixing means 7. Before the base part 3 can be moved relative to the connecting part 5, the fixation means 8 has to be first removed from the fixing unit 7.

### List of Reference Signs

1 fastening system

2 marker device (reference star)

3 base part

4 holding unit

5 connecting part (connecting pin)

6 fastening unit (through-hole)

7 fixing unit (through-hole)

8 fixation means (pin)

10 outer thread

11 translation limiting unit

12 clamping means

13 biasing means

14 releasing means

15 positioning means

16 inset

21, 22, 23 marker

51 connecting part (connecting pin)

61 radial clearance

81 fixation means (screw)

91, 92, 93 translation limiting unit

1-15. (canceled)

16. A medical fastening system for fastening a medical marker device to an anatomical body part, the fastening system comprising:
a) a base part comprising a holding unit for holding the marker device; and
b) a connecting part for connecting the base part to the anatomical body part,
c) wherein the base part comprises a fastening unit comprising a clamping unit configured to releasably clamp the connecting part so as to fasten the connecting part to the base part so that the base part remains rotatable relative to the connecting part,
d) and wherein the base part comprises a fixing unit for fixing the base part to the anatomical body part,
e) wherein the medical fastening system further comprises a fixation device which can be inserted into the fixing unit so as to fix the position of the base part relative to the anatomical body part and to prevent operation of the clamping means for releasing the connecting part while the fixation device is inserted in the fixing unit.

17. The fastening system according to claim 16, wherein the fixing unit comprises at least one of a through-hole, a blind hole and a thread hole.

18. The fastening system according to claim 16, wherein the fastening unit and/or the connecting part is configured to keep the base part translatable relative to the connecting part after the connecting part has been fastened to the base part.

19. The fastening system according to claim 16, wherein the connecting part comprises a positioning unit and wherein the fastening unit comprises a clamping unit which is configured to interact with the positioning unit for fixing the position of the fastening unit relative to the connecting part in the longitudinal direction of the connecting part.

20. The fastening system according to claim 19, wherein the fastening unit comprises a releasing unit for releasing the fastening unit from the connecting part.

21. The fastening system according to claim 16, wherein the fastening unit comprises at least one of a through-hole, a thread hole and a blind hole.

22. The fastening system according to claim 16, wherein the connecting part is configured to be attached to the marker device.

23. The fastening system according to claim 22, wherein the connecting part comprises a resisting unit for resisting a force applied to the marker device after it has been attached to the connecting part.

24. The fastening system according to claim 16, wherein the connecting part is a longitudinally-shaped structure, in particular at least one of a screw, nail and pin.

25. The fastening system according to claim 24, wherein the connecting part comprises an exterior thread and the fastening part comprises a corresponding interior thread.

26. The fastening system according to claim 16, wherein the connecting part comprises at least one translation limiting means which limits translatability of the base part relative to the connecting part.

27. The fastening system according to claim 16, wherein the base part comprises a polymer material.

28. The fastening system according to claim 16, wherein the connecting part comprises a metal material.

29. A medical system, comprising:
a) the fastening system according to claim 1; and
b) the medical marker device.
